(19) United States
(12) Patent Application Publication
(10) Pub. No.: US 2023/0053446 A1
(43) Pub. Date: Feb. 23, 2023

(54) ACTIVE BI-DIRECTIONAL OPEN PATH GAS DETECTION SYSTEM

(71) Applicant: Rosemount Inc., Shakopee, MN (US)

(72) Inventors: Tsviel BOUHBUT, Be'er Sheva (IL); Yossi Ben-Aderet, Zikim (IL); Dimitriy Grigorovitch, Be'er Sheva (IL); Uzziel Sheintop, Haggay (IL)

(21) Appl. No.: 17/708,743

(22) Filed: Mar. 30, 2022

Related U.S. Application Data
(60) Provisional application No. 63/234,813, filed on Aug. 19, 2021.

(51) Int. Cl. G01N 21/3504; G01N 33/00; H04B 1/3827; H04B 1/40

(52) U.S. Cl. CPC G01N 21/3504; G01N 33/0073; G01N 33/0027; H04B 1/3827; H04B 1/40

(57) ABSTRACT

An open path gas detection system includes a transmitter and a receiver. The transmitter is configured to generate illumination across an open path. The receiver is positioned to detect the illumination from the transmitter after the illumination has passed through the open path and detect a gas of interest based on the illumination. However, the laser can also be used for gas detection systems in other circumstances. The transmitter and receiver are configured to communicate wirelessly. A method of operating an open path gas detection system is also provided.

### CROSS-REFERENCE TO RELATED APPLICATIONS

The present application is based on and claims the benefit of U.S. Provisional Patent Application Ser. No. 63/234,813 filed Aug. 19, 2021, the content of which application is hereby incorporated by reference in its entirety.

### BACKGROUND

Open Path Gas Detectors (OPGD) are line-of-sight gas monitors commonly installed to monitor for gas presence over long distances. Open Path Gas Detectors provide a high speed of response, they operate in extreme conditions, and require fewer instruments to monitor large areas. These detectors generally, detect the unique spectral fingerprint of an individual chemical substances. Such gas detectors typically consist of a pair of devices; a source unit and a detector unit. The source unit generates a high energy beam electromagnetic energy. The target gas absorbs some of the electromagnetic energy and transmits the rest. The detector unit then detects the transmitted energy at specific spectral ranges, based on the target gas.

Some open path gas detection systems employ wired communication between source unit (transmitter) and the detector unit (receiver). Such wired approaches require complicated infrastructure, complex installation, cables wear and tear and require frequent maintenance. Furthermore, with that kind of system interface, the wired communication is used to obtain information regarding the units (transmitter and receiver) themselves and not information regarding the medium/atmosphere between the units—which is particularly important data when talking about open path gas detection systems. Having feedback from transmitter and receiver and the path between them is necessary in order to obtain clear understanding of system surroundings, which is not possible when using wired communication.

### SUMMARY

An open path gas detection system includes a transmitter and a receiver. The transmitter is configured to generate illumination across an open path. The receiver is positioned to detect the illumination from the transmitter after the illumination has passed through the open path and detect a gas of interest based on the illumination. The transmitter and receiver are configured to communicate wirelessly. A method of operating an open path gas detection system is also provided.

### BRIEF DESCRIPTION OF THE DRAWINGS

FIG. 1 is diagrammatic view of a unidirectional open path gas detection system having a passive receiver.

FIG. 2 is a diagrammatic view of two open path gas detection systems illustrating the problem of crosstalk.

FIG. 3 is a diagrammatic view of an active bi-directional open path gas detection system in accordance with one embodiment.

FIGS. 4 and 5 are side elevation and perspective views of a laser module for an active bi-directional open path gas detection system in accordance with an embodiment of the present invention.

FIG. 6 is a diagrammatic view of an experimental setup illustrating efficacy of an active bi-directional open path gas detection system in accordance with an embodiment of the present invention.

FIG. 7 is a system block diagram of a bi-directional open path gas detection system in accordance with an embodiment of the present invention.

FIG. 8 is a flow diagram of a method of operating an active, bi-directional open path gas detection system in accordance with an embodiment of the present invention.

### DETAILED DESCRIPTION OF ILLUSTRATIVE EMBODIMENTS

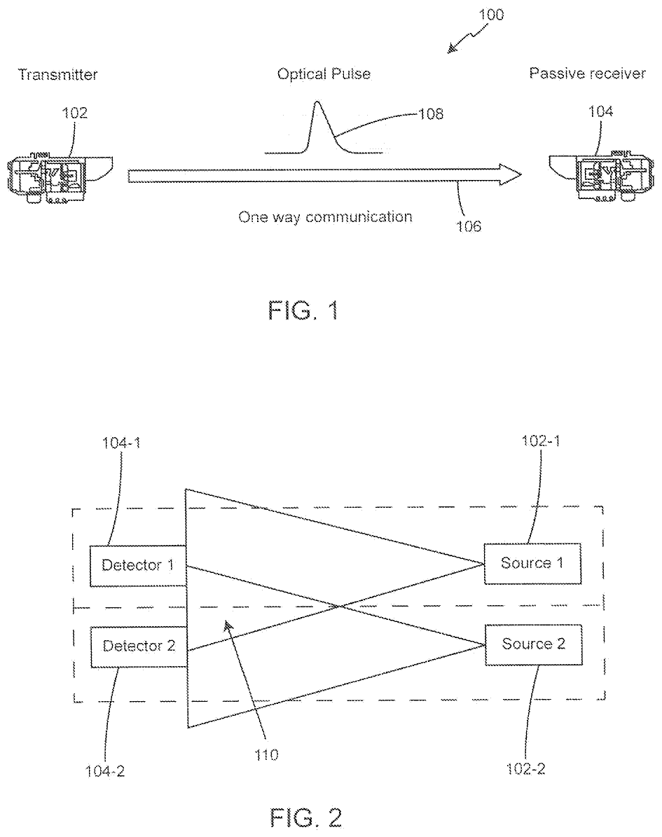

FIG. 1 is diagrammatic view of a unidirectional open path gas detection system. System 100 includes transmitter 102 and receiver 104, which are arranged such that an optical pulse 108 delivered from transmitter 102 will travel along path 106 to receiver 104. Receiver 104 receives the optical pulse and is configured to analyze the spectral characteristics of the received signal to determine whether the optical pulse passed through a gas of interest. As shown in FIG. 1, the communication from transmitter 102 to receiver 104 is entirely unidirectional. Thus, passive receiver 104 has no ability to communicate to transmitter 102. This limitation of system 100 can give rise to crosstalk in situations where multiple such systems 100 are used in proximity with one another. Open path gas detection systems that do not employ any kind of synchronization between the transmitter and receiver are known to suffer from issues such as crosstalk and have difficulties detecting gas during harsh environmental conditions. Crosstalk is a significant issue in open path gas detection systems. In terms of optical systems, “crosstalk” refers to the phenomenon by which a signal that is transmitted by the transmitter for its relevant receiver creates an undesired effect in receiver of a separate set nearby. Harsh weather conditions can also cause system difficulties since the receiver may be getting a much lower signal from the transmitter compared to normal operation. This can lead to faulty operation of the system as it affects the optical performance and the detection capabilities and will eventually cause the receiver to miss actual gas reading in the monitored path.

FIG. 2 is a diagrammatic view of two open path gas detection systems illustrating the problem of crosstalk. FIG. 2 illustrates a first open path gas detection system having source 102-1 and detector 104-1 operating in proximity to a second open path gas detection system having source 102-2 and detector 104-2. Each of the open path gas detection systems shown in FIG. 2 operates as described above with respect to system 100. However, the proximity of the two systems allows optical pulses or energy 110 from one source, such as source 102-1, to be detected by a different detector (104-2) than the intended detector (104-1). This detection of erroneous optical pulses or energy is known as cross-talk and can cause errors in the affected systems.

In order to have authentic information about system environment the interface between transmitter and receiver should be optical and wireless. Essentially, it is desirable to provide an open path gas detection system with the ability to learn its environment continuously or at least periodically. Employing wireless communication presents the challenge of having optical radiation intensity above allowed limits, which may be a significant obstacle in applications which require optical intrinsically safety approval. In many applications where a long distance needs to be covered, the energy required to communicate optically over the long distance likely exceeds the limits of the standard, while the system still needs to comply to explosion proof regulations. In addition, high class lasers could expose a user in danger due to eye safety standard. At least some embodiments described herein provide an open path gas detection system that includes a source (transmitter) and detector (receiver) that can employ UV and/or IR electromagnetic energy for gas detection as well as employ laser IR energy for bi-directional communication between the source and the detector.

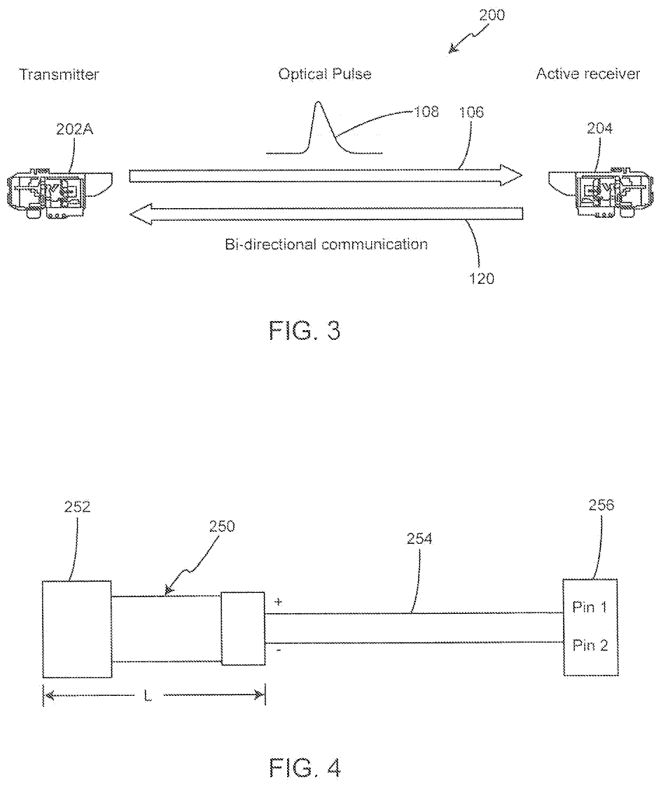

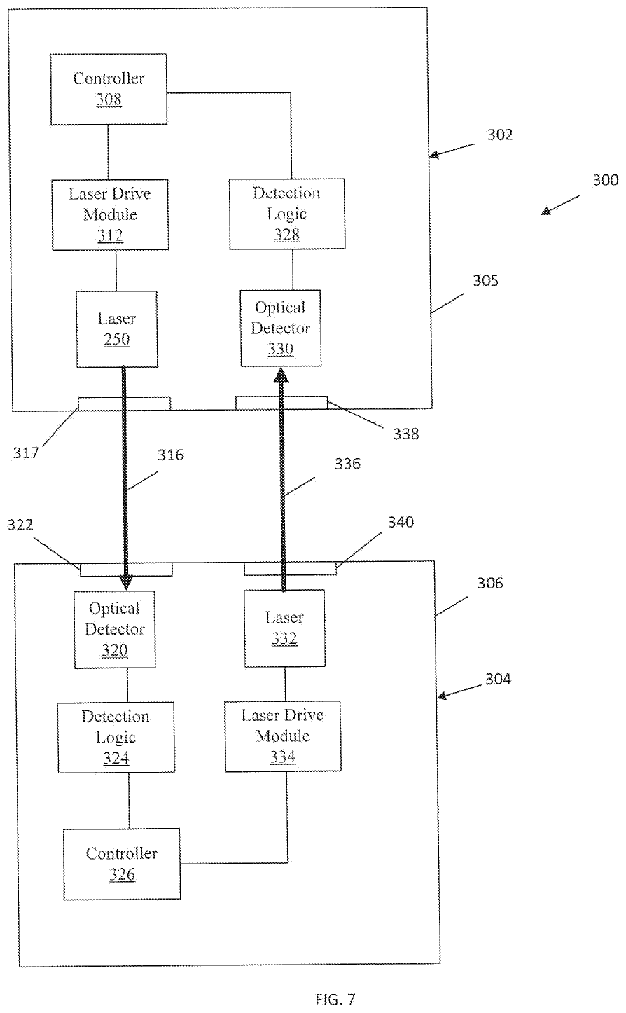

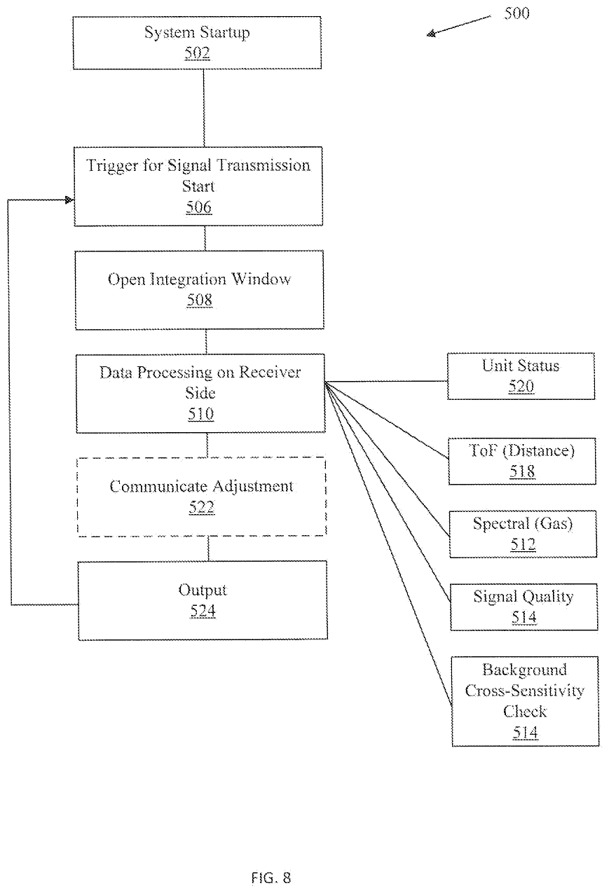

FIG. 3 is a diagrammatic view of an active, bi-directional open path gas detection system 200 in accordance with an embodiment of the present invention. System 200 includes a transmitter 202 and a receiver 204. Like transmitter 102, transmitter 202 is configured to send an optical pulse 108 along path 106 to receiver 104, which is configured to detect spectral attributes of the received pulse 108 in order to detect a gas of interest along path 106. However, in accordance with an embodiment of the present invention, receiver 204 includes an optical source (shown in FIG. 7) that allows receiver 204 to send an optical pulse, or other suitable signal or waveform, along path 120 to transmitter 202. In one embodiment, the optical source is a laser transmitter. Additionally, transmitter 202 also includes a detector and suitable detection logic to detect the optical pulse/signal from receiver 204. Adding an optical source on the receiver side allows the use of wireless communication between the two units, making the system active and bi-directional. In this way additional features and control options for the transmitter and receiver are enabled. Such new features include, without limitation, monitoring medium characteristics (harsh weather/fog/snow etc.) as part of an Environment Learning System, distance calculation (using time-of-flight (ToF) technology), pulse repetition frequency (PRF) increasing when transmitter signal is low as part of environmental compensation module, preventing crosstalk from nearby transmitters et cetera.

Providing an active optical source on the receiver side as well as detection electronics on the source side transforms an otherwise unidirectional system into an active bi-directional one, having the ability to communicate wirelessly, getting digital communication and spectral information from it. Additionally, bi-directional communication enables the two units to have increased awareness about the environment as well as the units themselves. This awareness can help provide the units with the ability to dynamically adjust working mode of the transmitter, such as when interference like heavy rain, fog, or a blizzard occur. Thus, adjusting the working mode of the units facilitates compensation for environmental conditions so that the receiver will be able to get viable signal from the transmitter, despite the harsh weather conditions, and the system will continue to operate normally, and detect gas.

Many of the environments in which open path gas detectors operate are highly volatile or explosive and could be ignited by a spark or elevated surface temperature in the gas detection system. Thus, for such gas detection systems, it is highly desirable to comply with explosion-proof ratings. Such ratings require that any explosion or flame generated within a complying electrical device will not ignite the environment of the device. These ratings drive such design constraints as housing wall thickness and material and the provision of a flame quenching pathway from an interior of the device to the external environment. One example of an explosion-proof rating is an ATEX certification to Ex-d standards EN60079-0 and EN60079-1 for potentially explosive atmospheres. Generally, explosion-proof housings are relatively bulky in order to be mechanically robust enough to contain an internal explosion without rupturing. Generally, such explosion-proof containers are very robust metal enclosures that are designed to withstand explosive pressures. However, for optical devices, the enclosure must accommodate a window of some sort in order to allow the illumination to pass through to the environment.

Another way to protect hazardous environments is to require that devices operating therein comply with intrinsic safety requirements. When the electronics are intrinsically safe, they inherently cannot generate the required temperature or spark to generate an explosion, even under fault conditions. An example of an intrinsic safety specification is the standard promulgated by Factory Mutual Research in October 1998 entitled APPROVAL STANDARD INTRINSICALLY SAFE APPARATUS AND ASSOCIATED APPARATUS FOR USE IN CLASS I, II, AND III, DIVISION 1 HAZARDOUS (CLASSIFIED) LOCATIONS, CLASS NUMBER 3610. Intrinsic safety requirements generally specify such low energy levels that compliance is simply not possible with circuitry that involves high voltages, high currents, and/or high wattage, such as AC circuits. In at least some embodiments described herein, the circuitry is designed and configured to comply with an intrinsic safety requirement, such as that set forth above.

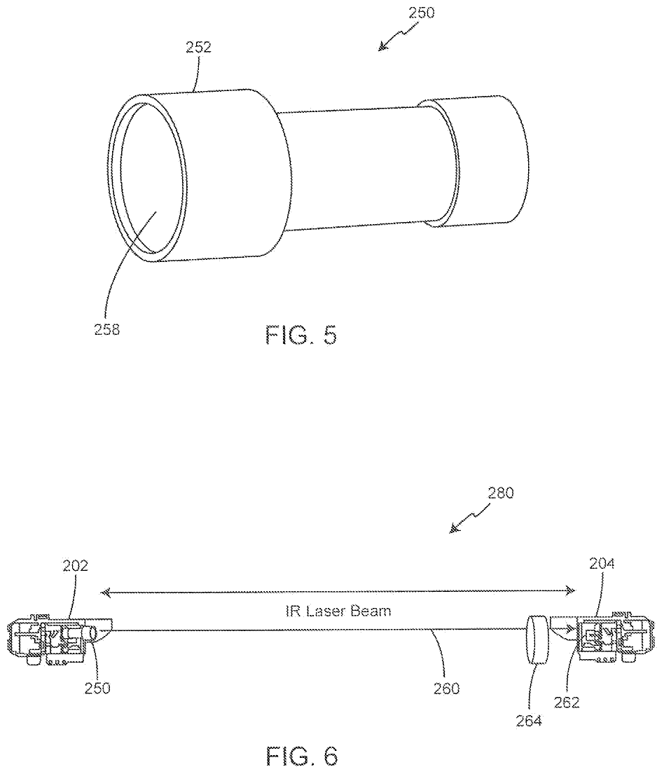

FIGS. 4 and 5 are side elevation and perspective views of a laser module for an active bi-directional open path gas detection system in accordance with an embodiment of the present invention. Providing a laser module that is compact helps reduce the cost of the system, which is of particular importance for explosion-proof systems. Further it is desirable to provide a laser module that has sufficient energy for low-energy long-distance communication and/or detection, but that can still comply with class 1 laser standards. Laser module 250 meets these criteria. Laser module 250 has a laser output end 252 that is shaped circularly and preferably has a diameter of about 8.5 mm. Laser output end 252 extends approximately 6 mm. The overall length “L” of laser module 250 is about 20 mm. Laser module 250 also includes a pair of twisted pair conductors 254 that extend to a 2-pin connector 256 that connects to suitable energization circuitry within the unit. As shown in FIG. 5, laser module 250 also generally includes an output lens 258. In one embodiment, laser module 250 is configured to generate an infrared (IR) laser beam.

FIG. 6 is a diagrammatic view of an experimental setup illustrating efficacy of an active bi-directional open path gas detection system in accordance with an embodiment of the present invention. System 280 includes a transmitter 202 provided with a laser module 250, described above with respect to FIGS. 4 and 5. Laser module 250 of transmitter 202 generates an IR laser beam 260 toward receiver 204. Receiver 204 includes a detector 262 that is configured to detect the IR laser beam. In order to simulate the attenuation caused by environmental conditions, such as rain or a blizzard, a ND filter 264 was placed in the path of beam 260. The experimental setup indicated successful operation at a detection distance of 200 meters even with a 90% obscuration from filter 264. This indicates a satisfactory signal-to-noise ratio and balanced design utilizing a compact laser module 250 that complies with class 1 laser standards.

FIG. 7 is a system block diagram of a bi-directional open path gas detection system in accordance with an embodiment of the present invention. System 300 includes a transmitter 302 and receiver 304. Each of transmitter 302 and receiver 304 preferably includes an explosion-proof housings 305, 306, respectively. Transmitter 302 includes a controller 308 coupled to laser drive module 310. Laser drive module 310 can include power handling components as well as frequency control and pulse generation logic such that upon receiving a signal from controller 308, laser drive module 312 is configured to cause laser module 250 to generate a suitable pulse or signal 316 toward receiver 304 through housing window 317. Transmitter 302 can employ any suitable laser module, but preferably employs laser module 250, described above. Controller 308 can be any suitable arrangement of circuitry or logic that is able to cause laser module 250 to generate a pulse or signal 316 as well as to employ communication module 314 to communicate with receiver 304 using wireless communication 318. In one embodiment, controller 308 is a microprocessor.

Receiver 304 includes an optical detector 320 positioned near detector window 322 in housing 306. Optical detector 320 is configured to generate an electrical signal representative of optical pulse or signal 316 passing through window 322. Optical detector 320 is coupled to detection logic module 324, which is configured to receive the electrical signal from optical detector 320 and amplify the signal as well as analyze spectral components of the received signal to provide an indication of the spectral composition of the signal to controller 326. In some embodiments, optical detector 320 and detection logic 324 may be combined in a single physical device. Controller 326 can be any suitable arrangement of circuitry or logic that is able to receive the spectral composition information from detection logic 324 and generate useful gas detection information based on the spectral composition information. In one embodiment, controller 326 is a microprocessor.

As shown in FIG. 7, controller 326 is coupled to laser drive module 334, which is coupled to laser 332. Laser 332 is configured to generate laser illumination through window 340 as indicated at reference numeral 336. Note, window 340 may be a separate window from window 320, or it may be the same component. The laser 336 travels to transmitter 302 where it passes through window 338 and is detected by optical detector 330. Note, window 338 may be a separate window from window 317, or it may be the same component. Optical detector 330 is coupled to detection logic 328, which provides information indicative of the detected signal to controller 308. In this way, receiver 304 is able to communicate wirelessly with transmitter 302 using laser communication. Thus, receiver 304 is able to transmit wireless information to and receive wireless information from transmitter 302. This bi-directional communication ability between transmitter 302 and receiver 304 enables a variety of new features and modes for open path gas detection sensor 300. For example, environmental conditions may attenuate some of laser pulse 316 such that the signal-to-noise ratio falls below a selected threshold as detected by receiver 304. When this occurs, receiver 304 communicates wirelessly to transmitter 302 to instruct transmitter 302 to increase the intensity of the laser beam.

One particular synergy of system 300 is that since each device is capable of transmitting and receiving an optical signal, bi-directional communication can be performed using the optical signal itself. Further, as can be appreciated, since each device can both generate and receive optical pulses or signals, the detection ability of system 300 is redundant in the event that one of a laser or detector should fail for a single unit.

In some embodiments, laser drive module 312 may be combined into the same physical component as the laser, such as compact laser source module 250 (shown in FIGS. 4 and 5). Additionally, detection logic, such as detection logic 328 and an optical detector, such as optical detector 330, may also be combined in a single physical device.

FIG. 8 is a flow diagram of a method of operating an active, bi-directional open path gas detection system in accordance with an embodiment of the present invention. Method 500 begins at block 502 where system startup occurs. System startup can include one or more self-tests for each of the transmitter or receiver. For example, a self-test may include verifying sufficient operating voltage, testing optical components (such as a laser or detector), et cetera. System startup 502 can also include a pairing operation to pair a receiver to its respective transmitter. The pairing can occur in any suitable manner. As part of the pairing process, or shortly thereafter, the receiver and the transmitter synchronize their respective timers. Next, at block 506, a trigger is generated for a signal transmission start. In one example, this trigger can be generated by the receiver instructing the transmitter to begin transmission of an optical pulse at a pre-defined timeframe. Note, embodiments of the present invention can also be practiced where the transmitter instructs the receiver of a pre-defined timeframe in which it will transmit the optical pulse. Regardless, as a result of block 506, the receiver will open its integration window for the pre-defined timeframe to receive the optical pulse or signal from the transmitter, as indicated at block 508.

At block 510, the receiver performs data processing on the received signal. The data processing can include analyzing the spectral characteristics of the received signal to detect one or more gasses of interest, as indicated at block 512. This analysis generates the primary output of the gas detection system. The spectral characteristics can also be stored and analyzed over time to identify environmental medium condition and/or status. Further, artificial intelligence and machine learning can be employed and trained on the spectral characteristics to predict future values or conditions. However, the receiver also analyzes the received signal in other regards to determine additional parameters of interest. For example, the signal strength or quality can be determined, as indicated at block 514. Variations in the signal strength or quality may provide an indication of signal degradation due to rain/fog/snow/mist. Further, the measuring the signal level during known periods of zero signal can provide an indication of background noise or cross-talk, as indicated at block 516. Additionally, since the receiver and transmitter have synchronized timers and the receiver knows the exact time at which the transmitter launches the optical pulse, the time it takes for the pulse to cross the open path can be measured by the receiver. This time-of-flight measurement 518 provides a direct indication of distance, which may be an important variable to monitor for a particular application. The receiver may also analyze the received signal to determine unit status for the transmitter and/or the receiver itself, as indicated at block 520.

As a result of the analysis performed at block 510, the receiver may determine that some aspect of the transmitted optical pulse needs to be adjusted. In such case, optional block 522 can be executed where the receiver transmits an adjustment request to the transmitter. For example, at block 510, the receiver may determine that signal strength has fallen below a low-signal threshold and that the intensity of the laser beam should be increased by a particular amount. This intensity increase command, as well as an intensity increase amount parameter can be communicated from the receiver to the transmitter at optional block 522. The transmitter will then responsively increase the laser beam intensity by the requested amount.

At block 524, the system output is provided. In embodiments, where the receiver/transceiver includes wireless communication circuitry, such as using WirelessHART communication protocol, the receiver/transceiver may report the output directly to a process communication network. Additionally, or alternatively, the system may provide the output directly on a display of the receiver/transceiver. Further, in situations where the gas detection level is above a certain threshold, the output may also include a visual or audible alarm. Finally, since the receiver and transmitter can communicate with one another, the output may also be communicated from the receiver to the transmitter to be displayed locally on a display/alarm of the transmitter as well. With the output generated, method 500 repeats by returning to block 506.

As set forth above, some of the embodiments described herein provide a small, low-energy, long-distance optical communication system for an open path gas detection system that complies with at least one explosion-proof specification, and/or complies with Class 1 Laser requirements. In some embodiments, wireless communication module is designed to prevent crosstalk between different pairs (pair/set means receiver and transmitter) using wireless communication between the receiver and the transmitter. By that, each receiver will identify and pair with the relevant transmitter to avoid crosstalk.

Although the present invention has been described with reference to preferred embodiments, workers skilled in the art will recognize that changes may be made in form and detail without departing from the spirit and scope of the invention. For example, while embodiments described herein generally provide a gas detection system with a number of additional parameters of interest, additional capabilities are also enabled, such as using the system to map the surrounding area using LIDAR technology.

What is claimed is:

1. An open path gas detection system comprising:
a transmitter configured to generate illumination across an open path; and
a receiver positioned to detect the illumination from the transmitter after the illumination has passed through the open path, the detector being configured to detect a gas of interest based on the illumination, wherein the transmitter and receiver are configured to communicate wirelessly.

2. The open path gas detection system of claim 1, wherein the wireless communication is optical communication over the open path.

3. The open path gas detection system of claim 1, wherein the wireless communication is bi-directional wireless communication.

4. The open path gas detection system of claim 3, wherein the open path gas detection system is configured to dynamically adjust a working mode of the transmitter based on illumination detected by the receiver.

5. The open path gas detection system of claim 4, wherein the working mode of the transmitter is adjusted based on environmental interference.

6. The open path gas detection system of claim 1, wherein the transmitter includes a compact laser module configured to generate an optical laser pulse along the open path to the receiver.

7. The open path gas detection system of claim 6, wherein the laser module is configured to generate an IR laser pulse.

8. The open path gas detection system of claim 1, wherein the receiver includes a compact laser module configured to generate an optical laser signal along the open path to the transmitter.

9. The open path gas detection system of claim 8, wherein the transmitter is configured to detect the optical laser signal from the receiver.

10. The open path gas detection system of claim 9, wherein the compact laser module complies with Class 1, laser standard.

11. The open path gas detection system of claim 1, wherein at least one of the transmitter and the receiver includes an explosion-proof housing.

12. A transceiver for detecting a gas along an open path, the transceiver comprising:
an explosion-proof housing;
a controller disposed within the explosion-proof housing;
a compact laser module disposed within the explosion-proof housing and operably coupled to the controller;
an optical detector operably coupled to the controller; and
wherein the controller is configured to communicate wirelessly with a remote transceiver to detect the gas along the open path.

13. The transceiver of claim 12, wherein the controller is configured to communicate wirelessly with the remote transceiver using optical communication via the compact laser module and the optical detector.

14. The transceiver of claim 12, wherein the compact laser module is a Class 1 laser device.

15. The transceiver of claim 12, wherein the compact laser module is configured to generate an IR laser beam along the open path.

16. The transceiver of claim 12, wherein the transceiver is configured to pair with the remote transceiver.

17. The transceiver of claim 16, wherein the transceiver and the paired remote transceiver have synchronized timers.

18. A method of operating an active, bi-directional open path gas detection system, the method comprising:
generating a signal transmission start that causes a transmitter to begin transmission of an optical pulse at a pre-defined timeframe;
opening an integration window of the receiver for the pre-defined timeframe;
obtaining an optical signal with the detector during the pre-defined timeframe;
analyzing the received optical signal to detect a gas based on spectral characteristics of the received optical signal; and
analyzing the received optical signal to obtain an additional parameter of interest; and
generating an output indicative of the detected gas.

19. The method of claim 18, wherein the additional parameter of interest is selected from the group consisting of: unit status; time-of-flight of the optical signal, signal quality, background noise, pulse repetition frequency, and cross-talk.

20. The method of claim 18, and further comprising causing the receiver to communicate an adjustment to the transmitter based, at least in part, on the additional parameter of interest.
